(19) United States
(12) Patent Application Publication
(10) Pub. No.: US 2006/0093652 A1
(43) Pub. Date: May 4, 2006

(54) Collagen product with an alginate sheath and method for producing the same

(75) Inventors: Wei-Hsin Lin, Panchiao City (TW); Ching-Hsiang Lu, Tu Chen City (TW)

Correspondence Address: THE WEBB LAW FIRM, P.C., 700 KOPPERS BUILDING, 436 SEVENTH AVENUE, PITTSBURGH, PA, 15219, US

(73) Assignee: Taiwan Textile Research Institute

(21) Appl. No.: 11/082,393

(22) Filed: Mar. 17, 2005

(30) Foreign Application Priority Data
Nov. 4, 2004 (TW) 93133652

(51) Int. Cl. A61K 9/70; C07K 14/78

(52) U.S. Cl. 424/443; 530/356

(57) ABSTRACT

A collagen product with an alginate sheath and a method for producing the same are described. A first solution containing at least an alginate is prepared. A second solution containing collagen and at least an alkaline earth metallic salt is then prepared. The second solution is spun through a spinneret in the first solution to obtain a collagen product with an alginate sheath. The collagen product with an alginate sheath is dehydrated with hydrophilic solvent.

### RELATED APPLICATIONS

The present application is based on, and claims priority from, Taiwan Application Serial Number 93133652, filed on Nov. 4, 2004, the disclosure of which is hereby incorporated by reference herein in its entirety.

### BACKGROUND

1. Field of Invention

The present invention relates to a collagen product, and more particularly to a composite product made of alginate and collagen and a method for forming the same.

2. Description of Related Art

Medical devices made of different materials, such as alginate, collagen, chitin or PU, have different functions in medical treatment. For example, calcium alginate promotes hemostasis and healing; chitin is anti-microbial and provides protection from wound infection.

Collagen has been used in medical treatment and cosmetics. One benefit of collagen is that it is good for tissue regeneration and repair because collagen is a key component of tissue and organs, such as connective tissue and the epidermis. Another benefit is that the collagen extracted from animal tissue does not induce an immune response because the similar structures of human and animal collagens. Yet another benefit is that collagen is abundant in bones, skin, ligament of animals, and easily absorbed and digested in human bodies. Therefore, collagen extracted from animal tissue has been applied on medical devices such as sutures, wound dressings, or repair materials for cartilage and ligament.

The precipitation method is a typical method for forming collagen fibers. In the precipitation method, water-soluble collagen is precipitated in high concentration metal salt solution and spun into fibers. Then, the salts and organic solvent are washed from collagen fibers with water. During the washing step, another organic solvent is added in order to avoid losing the collagen. The collagen fiber obtained by the foregoing methods is easy to absorb water, and swells or deliquesces in a humid environment. Consequently, products made of the conventional collagen fibers have to be packaged with multi-layer tight pack for damp proof.

Another method for forming a collagen fiber comprises steps of obtaining collagen fiber by the precipitation method, and then immersing the collagen fiber in a solution containing cross-linking agents (such as formaldehyde or glutaraldehyde) to form cross-linked structure in the collagen (U.S. Pat. No. 6,160,096). Although the cross-linking agent can improve the deliquescence resistance by forming a cross-linked structure, the cross-linking agent is toxic and not easily removed from the collagen fiber. Therefore, the collagen fibers formed by the methods described above are only suitable for external uses like wigs, but not appropriate for medical or cosmetic use.

### SUMMARY

It is therefore an aspect of the present invention to provide a collagen fiber with moisture resistance to overcome the swelling and deliquescence of conventional collagen fibers.

It is another aspect of the present invention to provide a collagen fiber with good anti-deliquescence to reduce amount of packaging of the collagen fiber to protect the collagen fiber from moisture.

It is yet an aspect of the present invention to provide a collagen fiber with good anti-deliquescence and without toxic residues.

It is still an aspect of the present invention to provide a collagen membrane with good anti-deliquescence and a method for forming the same.

In accordance with the foregoing and other aspects of the present invention, a collagen product with an alginate sheath is provided. The collagen product has a collagen core, and the core is sheathed with alkaline earth metal alginate. Therefore, the core made of collagen avoids swelling and deliquescing in a humid environment.

In accordance with the foregoing and other aspects of the present invention, a method for forming the collagen product with an alginate sheath is provided. At least an alginate is dissolved in a solvent to obtain a first solution. A second solution containing collagen and at least an alkaline earth metal salt is prepared. The second solution is injected into the alkali metal alginate solution to form a collagen product with an alginate sheath. After dehydrating the collagen product with an optional, second hydrophilic solvent, the collagen product with an alginate sheath, according to the present invention, are obtained.

The concentration of the alginate in the first solution is about 0.05% to 5% by weight, and preferably about 2% to 5% by weight. The solvent for the first solution is water or water containing a first hydrophilic solvent, where the concentration in weight percent of the first hydrophilic solvent is about 0% to 30%. In the second solution, the concentration in weight percent of the collagen is about 0.1% to 20%, and preferably about 1% to 6%; the concentration in weight percent of the alkaline earth metal salt is about 0.01% to 15%, and preferably about 2% to 5%. Moreover, the alkaline earth metal alginate sheath is made by reacting an alginate with an alkaline earth metal salt, where the alkali earth salt is a soluble salt such as, for example, beryllium, magnesium, calcium, strontium, barium or any combination thereof.

### BRIEF DESCRIPTION OF THE DRAWINGS

The other aspects and features of the present invention can be more fully understood by the following description of preferred embodiments and accompanying drawings, in which

FIG. 1 is a flow-chart, illustrating a method for producing a collagen fiber with an alginate sheath according to the present invention;

FIG. 2 is a flow-chart, illustrating another method for producing a collagen fiber with an alginate sheath according to the present invention;

FIG. 3 is a flow-chart, illustrating a modified method for producing a collagen membrane with an alginate sheath according to the present invention; and

FIG. 4A-4C are SEM pictures, showing the structure of the surface and the cross-section of the collagen fiber with an alginate sheath according to an embodiment of the present invention.

### DESCRIPTION OF THE PREFERRED EMBODIMENTS

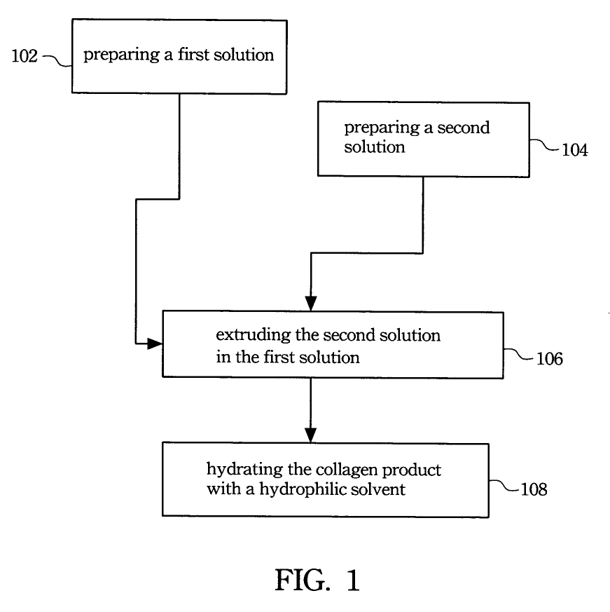

The following are several preferred embodiments for showing features of the collagen product with an alginate sheath and the methods for forming the same according to the present invention, wherein the collagen product can be, such as a collagen fiber or a collagen membrane with an alginate sheath. Referring to FIG. 1, in step 102, a first solution is obtained by dissolving at least an alginate with water or water containing a first hydrophilic solvent. According to steps 104, collagen and at least an alkaline earth metal salt is dissolved with water or other solvents to obtain a second solution. In step 106, the second solution is injected into the alkali metal alginate solution in the form of a fiber or a membrane through a spinneret to form a collagen product with an alginate sheath. As the optional step 108, the collagen product with an alginate sheath may be dehydrated with a second hydrophilic solvent.

### The First Embodiment

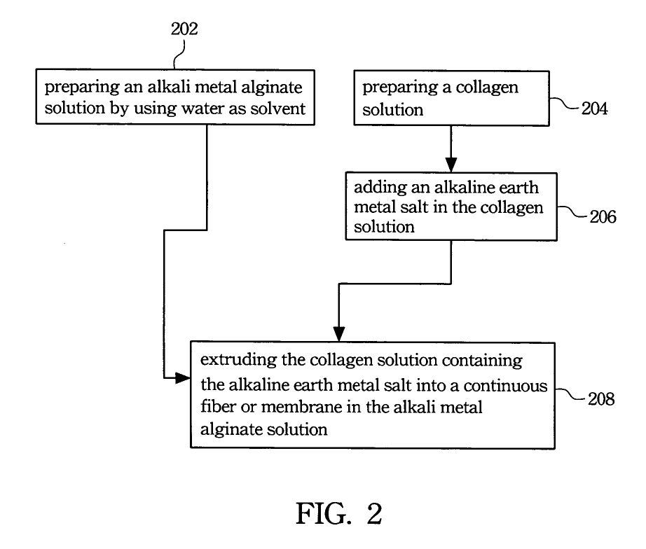

FIG. 2 is a flow chart illustrating a method for forming an alginate/collagen fiber according to the present invention. In step 202, an alkali metal alginate, such as sodium alginate, was dissolved in water to form a 2% (w/w) alkali metal alginate solution. In step 204, collagen was dissolved in water to form a 1% (w/w) collagen solution. In step 206, an alkaline earth metal salt such as calcium chloride (CaCl₂) was added to the collagen solution, and stirred to achieve a concentration of the alkaline earth metal salt of 2% by weight.

According to step 208, the collagen solution containing the alkaline earth metal salt was injected into the alkali metal alginate solution in the form of a continuous fiber by a spinning device, such as a spinneret. When the collagen solution contacted the alkali metal alginate solution, the alkaline earth metal salt in the collagen solution reacted with the alkali metal alginate to form alkaline earth metal alginate, such as calcium alginate, immediately sheathing the collagen solution. In this manner, a collagen fiber having a collagen core and an alkaline earth metal alginate sheath was obtained according to the present invention.

### The Second Embodiment

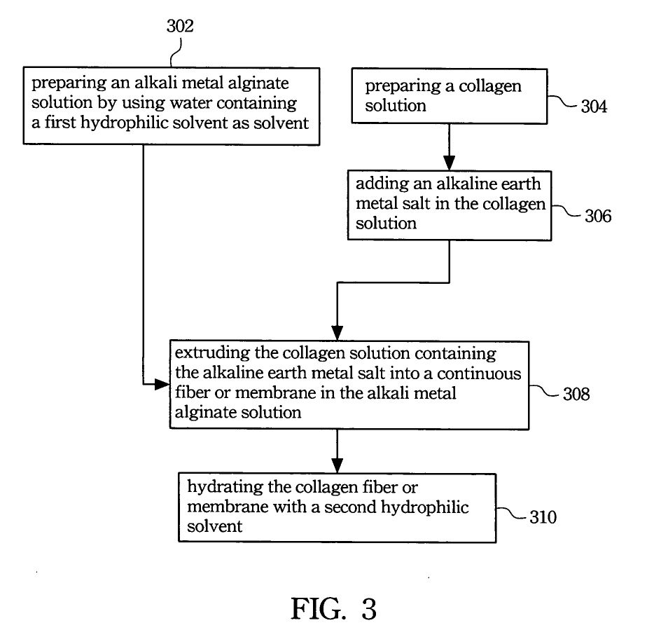

Referring to the flow-chart shown in FIG. 3, In step 302, an alkali metal alginate was dissolved in water containing a first hydrophilic solvent to form a 2% (w/w) alkali metal alginate solution, where the alkali metal alginate was, for example, potassium alginate, and the first hydrophilic solvent was, for example, ethanol. In step 304, collagen was dissolved in water to form a 6% (w/w) collagen solution. In step 306, an alkaline earth metal salt such as calcium chloride was added to the collagen solution, and stirred to achieve a concentration in weight percent of the alkaline earth metal salt of 5%.

According to step 308, the collagen solution containing the alkaline earth metal salt was injected into the alkali metal alginate solution by a spinning device, such as a spinneret. When the collagen solution contacted the alkali metal alginate solution, the alkaline earth metal salt in the collagen solution reacted with the alkali metal alginate to form alkaline earth metal alginate, such as calcium alginate, immediately sheathing the collagen solution. In this manner, a collagen fiber having a collagen core and an alkaline earth metal alginate sheath was obtained according to the present invention.

Further performing an optional step 310, the collagen fiber with the alkaline earth metal alginate sheath was immersed in a second hydrophilic solvent, such as acetone. Water in the collagen fiber with the alkaline earth metal alginate was removed by the vaporization of the second hydrophilic solvent.

### The Third Embodiment

The second embodiment was modified to obtain the third embodiment. Still referring to the flow-chart shown in FIG. 3, in step 302, an alkali metal alginate was dissolved in water containing a first hydrophilic solvent to form a 5% (w/w) alkali metal alginate solution, where the alkali metal alginate was, for example, sodium alginate or potassium alginate, and the first hydrophilic solvent was, for example, ethanol. The concentration in weight percent of the first hydrophilic solvent was about 0% to 30%. In step 304, collagen was dissolved in water to form a 15% (w/w) collagen solution. In step 306, an alkaline earth metal salt (such as calcium chloride) was added to the collagen solution, and stirred to achieve a concentration in weight percent of the alkaline earth metal salt of 10%.

According to step 308, the collagen solution containing the alkaline earth metal salt was injected into the alkali metal alginate solution in the form of a membrane by a spinning device. When the collagen solution contacted the alkali metal alginate solution, the alkaline earth metal salt in the collagen solution reacted with the alkali metal alginate to form alkaline earth metal alginate, such as calcium alginate, immediately sheathing the collagen solution. In this manner, a collagen membrane having a collagen core and an alkaline earth metal alginate sheath was obtained according to the present invention.

Further performing an optional step 310, the collagen membrane with the alkaline earth metal sheath was immersed in a second hydrophilic solvent, such as ethanol. Water in the composite membrane was removed by the vaporization of the second hydrophilic solvent.

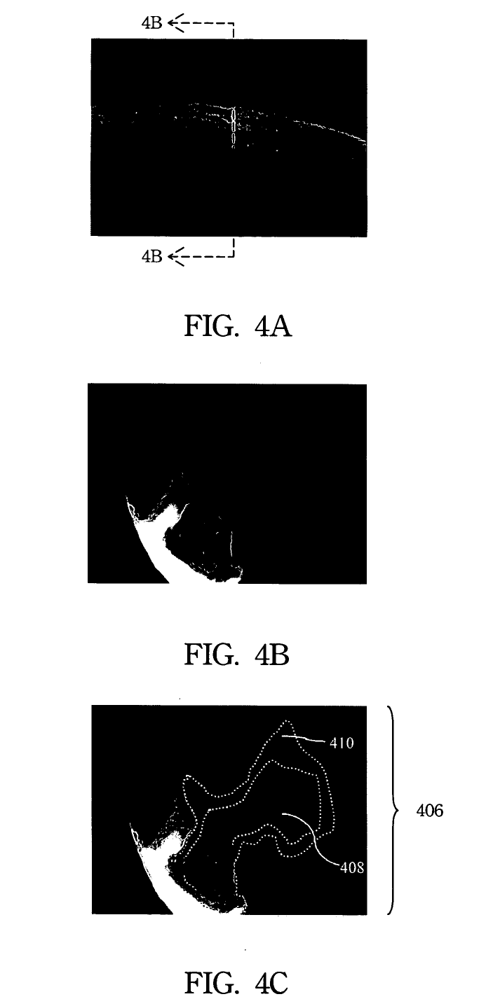

The collagen fiber with an alginate sheath according to the foregoing embodiment of the present invention had a surface structure shown in FIG. 4A, and a cross-sectional structure shown in FIG. 4B. FIG. 4B is a SEM picture illustrating that the fiber has a collagen core 408 sheathed with alkaline earth metal alginate 410.

There are some modifications according to the foregoing embodiments. The concentration of the alkali metal alginate solution is about 0.05% to 5% by weight, and preferably about 2% to 5% by weight. In the collagen solution, the concentration in weight percent of collagen is about 0.1% to 20%, and preferably about 1% to 6%. The concentration of the alkaline earth metal salt is about 0.01% to 15% by weight, and preferably about 2% to 5% by weight. The alkaline earth metal salt may be any water-soluble alkaline earth metal salt, such as beryllium, magnesium, calcium, strontium, barium and any combination thereof.

Anti-Deliquescence Test

The collagen fiber with the alkaline earth metal alginate sheath according to the foregoing embodiment of the present invention and a conventional collagen fiber formed by a precipitation method were tested by the following method for anti-deliquescence.

The collagen fiber with the alkaline earth metal alginate sheath according to the present invention and the conventional collagen fiber were weighed to obtain the dry weight thereof. Then, the two fibers were immersed in 10 ml distilled water and stirred at the same speed and at room temperature, respectively. During stirring, the two fibers were observed for structural integrity. Removing distilled water after 4 hours, the fibers were wiped with dry filter papers, and weighed for the wet weight thereof. The water absorption ratio of the collagen fiber with the alkaline earth metal alginate sheath and the conventional collagen fiber were calculated by the following function and shown in Table 1.
Water Absorption Ratio=(Wet weight−Dry weight)÷(Dry weight)

TABLE 1

| | |
|---|---|
| Conventional collagen | Alginate/collagen |
| fiber | fiber |

Before test

| | | |
|---|---|---|
| Dry weight (g) | 0.05 | 0.025 |
| Structural integrity | yes | yes |

After test

| | | |
|---|---|---|
| Wet weight (g) | N | 0.326 |
| Structural integrity | no | yes |
| Water absorption ratio | N | 12 |

N: unmeasurable

According to the result of the anti-deliquescence test, the conventional collagen fiber swelled and dissolved in the water rapidly, and therefore the water absorption ratio was unmeasurable. Due to the rapid swelling and deliquescence, the conventional fiber has some drawbacks. For instance, any medical product made of the conventional collagen fibers, such as a wound dressing, may swell and deliquesce after absorbing wound exudates.

The collagen fiber with an alginate sheath, according to the present invention, comprises a collagen core and an alginate sheath. The core was sheathed with the alginate to protect the collagen from deliquescence in humid environment.

The collagen fiber with an alginate sheath according to the present invention has an alginate sheath over the collagen core to protect the collagen core from deliquescence. Therefore, the collagen fiber with an alginate sheath according to the present invention remains intact in the water or after absorbing moisture from a humid environment. Furthermore, the collagen fiber with an alginate sheath according to the present invention is suitable for medical use, such as wound dressings, hemostatic and sutures, because no toxic residues are included in the composite fiber.

### CONCLUSION

According to the foregoing embodiments shown above, the present invention has several advantages. One is the improvement of the anti-deliquescence of the collagen product with an alginate sheath. The alkaline earth metal alginate sheath of the collagen product with an alginate sheath according to the present invention protects the collagen core from swelling and deliquescing in water or in a humid environment.

Another advantage is the low packaging cost of the collagen product with an alginate sheath according to the present invention. There is no need for multiple packages to protect the collagen product with an alginate sheath from moisture.

Yet another advantage is no toxic residues in the collagen product with an alginate sheath according the present invention, because the collagen product with an alginate sheath is formed without using toxic agents, like formaldehyde or glutaraldehyde, as cross-linking agents.

Still another advantage is that the collagen product with an alginate sheath is a good medical or cosmetic material. Alginate is a natural, biodegradable polymer with good biocompatibility. When the alginate sheath is made of calcium alginate, the collagen product with an alginate sheath according to the invention further provides hemostatic for a bleeding wound.

The present invention provides a collagen product with an alginate sheath that promotes hemostasis, absorbs liquid, and lower packages, non-toxic and anti-deliquescent by the combination of alginate and collagen. The present invention also provides a method for forming a collagen product without using a toxic cross-linking agent.

The specific conditions cited in the foregoing embodiments, such as concentration, temperature and time, are used merely for the purpose of representation, and shall not be used to restrict the scope of the present invention. It is to be understood that all modifications and equivalent variations in details or configurations made by anyone skilled in the art according to the principles disclosed above shall be included in the spirit and intent of the present invention.

1. A method for producing a collagen composite fiber or a collagen composite membrane, comprising:
preparing a first solution containing at least an alginate;
preparing a second solution containing collagen and at least one alkaline earth metal salt; and
extruding the second solution in the first solution to form a collagen fiber or a collagen membrane with an alginate sheath.

2. The method of claim 1, wherein a concentration of the alginate in the first solution is between about 0.05% and about 5% by weight.

3. The method of claim 2, wherein the concentration of the alginate in the first solution is between about 2% and about 5% by weight.

4. The method of claim 1, wherein the alginate in the first solution is sodium alginate, potassium alginate or any combination thereof.

5. The method of claim 1, wherein a concentration of the collagen in the second solution is between about 0.1% and about 20% by weight.

6. The method of claim 1, wherein a concentration of the collagen in the second solution is between about 1% and about 6% by weight.

7. The method of claim 1, wherein a concentration of the alkaline earth metal salt in the second solution is between about 0.01% and about 15% by weight.

8. The method of claim 1, wherein a concentration of the alkaline earth metal salt in the second solution is between about 2% and about 5% by weight.

9. The method of claim 1, wherein the alkaline earth metal salt is a soluble salt, and an alkaline earth metal of the alkaline salt is selected from the group consisting of beryllium, magnesium, calcium, strontium, barium and any combination thereof.

10. The method of claim 1, wherein a solvent of the first solution is water or water containing a first hydrophilic solvent.

11. The method of claim 10, wherein a concentration in weight percent of the first hydrophilic solvent is smaller than 30%.

12. The method of claim 10, wherein the first hydrophilic solvent is methanol, ethanol or acetone.

13. The method of claim 1, wherein a spinneret is used to extrude the second solution in the first solution to form the collagen fiber with an alginate sheath.

14. The method of claim 1, further comprising dehydrating the collagen fiber or the collagen membrane with an alginate sheath after the extruding step.

15. The method of claim 14, wherein a second hydrophilic solvent is used to dehydrate the collagen fiber or the collagen membrane with an alginate sheath.

16. The method of claim 15, wherein the second hydrophilic solvent is methanol, ethanol or acetone.

17. (canceled)

18. A collagen composite fiber or a collagen composite membrane, comprising:
a core at least made of collagen; and
a sheath at least made of alkaline earth metal alginate, wherein the core is jacketed by the sheath directly.

19. The collagen composite fiber or the collagen composite membrane of claim 18, wherein the alkaline earth metal of the alkaline earth metal alginate is selected from a group consisting of beryllium, magnesium, calcium, strontium, barium and any combination thereof.

20. (canceled)
